(19) United States
(12) Patent Application Publication
(10) Pub. No.: US 2014/0066704 A1
(43) Pub. Date: Mar. 6, 2014

(54) STEREOSCOPIC METHOD FOR MINIMALLY INVASIVE SURGERY VISUALIZATION

(71) Applicant: Vantage Surgical Systems Inc., Los Angeles, CA (US)

(72) Inventors: Mark BLUMENKRANZ, Portola Valley, CA (US); Steven SCHWARTZ, Los Angeles, CA (US); Jean-Pierre HUBSCHMAN, Beverly Hills, CA (US); Vacit ARAT, La Canada Flintridge, CA (US); Jason WILSON, Santa Ana, CA (US)

(21) Appl. No.: 14/014,164

(22) Filed: Aug. 29, 2013

Related U.S. Application Data
(63) Continuation-in-part of application No. 13/290,594, filed on Nov. 7, 2011.
Application No. 13/290,594 is a continuation-in-part of application No. 13/268,071, filed on Oct. 7, 2011, now abandoned.
Application No. 13/268,071 is a continuation-in-part of application No. 13/164,671, filed on Jun. 20, 2011.
Application No. 13/164,671 is a continuation-in-part of application No. 13/169,076, filed on Jun. 27, 2011.
Application No. 13/169,076 is a continuation-in-part of application No. 13/169,072, filed on Jun. 27, 2011.
(60) Provisional application No. 61/694,678, filed on Aug. 29, 2012.
(60) Provisional application No. 61/390,820, filed on Oct. 7, 2010.
(60) Provisional application No. 61/356,150, filed on Jun. 18, 2010.
(60) Provisional application No. 61/358,793, filed on Jun. 25, 2010.
(60) Provisional application No. 61/358,780, filed on Jun. 25, 2010.

(51) Int. Cl. A61B 1/00; A61B 17/34; A61B 19/00; A61B 1/04; A61M 13/00; A61B 17/00; A61B 1/06; A61B 1/313; A61B 1/015

(52) U.S. Cl. CPC A61B 1/00154; A61B 1/3132; A61B 17/3423; A61B 19/5202; A61B 1/015; A61M 13/003; A61B 17/00234; A61B 1/0676; A61B 1/042; A61B 1/00052; A61B 1/0005; A61B 1/00009; A61B 1/00039
USPC 600/103; 600/114; 600/111

(57) ABSTRACT

Embodiments of the invention are directed to minimally invasive surgical instruments and procedures using those instruments wherein the instruments include optical elements (e.g. a percutaneous optical channel (POC), a retaining plug and a stereoscopic imaging device (e.g. a stereoscopic camera) and a stereoscopic display device. The instruments be part of a system used to provide a surgeon with an indirect stereoscopic view of a surgical area undergoing a minimally invasive surgical procedure in a way that is perceived by the surgeon as being similar to that of an open surgery and providing a natural and intuitive environment to perform the surgery with a high degree of control.

### RELATED APPLICATIONS

This application claims priority to U.S. Provisional Patent Application No. 61/694,678, filed on Aug. 29, 2013, and is a Continuation-in-Part of U.S. patent application Ser. No. 13/290,594, filed on Nov. 7, 2011 which claims priority to U.S. Non-Provisional patent application Ser. No. 13/268,071, filed on Oct. 7, 2011 which claims priority to U.S. Provisional Patent Application No. 61/390,820, filed on Oct. 7, 2010, and U.S. Non-provisional patent application Nos. 13/164,671, 13/169,072, and 13/169,076 filed respectively on Jun. 20, 2011, Jun. 27, 2011, and Jun. 27, 2011; the '671 application claims benefit of U.S. Provisional Patent Application No. 61/356,150, filed on Jun. 18, 2010; both the '072 application and the '076 application are Continuation in Part Applications of the '671 application, while the '072 application claims priority to U.S. Provisional Patent Application No. 61/358,780, filed on Jun. 25, 2010 and the '076 application claims benefit of 61/358,793, filed on Jun. 25, 2010. These applications are incorporated herein by reference as if set forth in full herein.

### FIELD OF THE INVENTION

The present invention relates generally to the field of surgical instruments and surgical procedures, more particularly, to minimally invasive surgical instruments and minimally invasive surgical procedures involving visualization systems comprising specialized lenses that are placed in proximity to a surface of the body over which a minimally invasive surgical procedure can be performed.

### BACKGROUND OF THE INVENTION

Open surgery has the advantage of providing surgeons a direct and full 3-D view of the surgical field and their tools, all laid out in exactly the direction they are looking, providing a natural and intuitive environment to perform the surgery with a high degree of control. Minimally invasive surgery (MIS) is different; while it has well-known advantages in terms of less trauma and quick recovery time, it has the disadvantage that the direct view of the surgical field is not available to the surgeons who have to rely on endoscopes to provide the images of the surgical field. These images are generally: (1) Displayed only on electronic displays the location of which is most likely not the direction the surgeon's tools are actually pointing at; (2) Not stereoscopic (i.e. 2-D with no depth perception); and (3) Often too close, such that the tools, or sufficient portions of the tools, may not be in view at all times.

This results in a non-ideal and counterintuitive experience which necessitates substantial training for surgeons to get used to. As a consequence of the foregoing, it is desired to have minimally invasive visualization systems and methods that can overcome one or more of the aforementioned limitations.

### SUMMARY OF THE INVENTION

The foregoing needs are met, to a great extent, by the present invention, wherein some embodiments are intended to address one or more of the above noted fundamental problems associated with visualization systems used in conventional minimally invasive surgery. The Improved visualization methods and system of the various embodiments of the invention are applicable to many types of minimally invasive surgery, for example in the areas of thoracoscopic, laparoscopic, pelviscopic, arthroscopic surgeries. For laparoscopic surgery, significant utility will be found in cholecystectomy, hernia repair, bariatric procedures (bypass, banding, sleeve, or the like), bowel resection, hysterectomy, appendectomy, gastric/anti-reflux procedures, and nephrectomy.

In some aspects of the disclosure one or more of these problems are addressed by providing a practitioner, typically a surgeon, enhanced indirect or direct stereoscopic views of a surgical area. In some embodiments providing indirect views, a stereoscopic video camera can be able to obtain stereoscopic images via a single objective lens thus allowing for more light and higher spatial resolution. A stereoscopic display may be included and may be moved to an ergonomically correct sterile location while allowing for direct line of sight positioning of the stereoscopic camera and autostereoscopic (glasses-less) 3D visualization. In addition, an ancillary benefit of the monitor repositioning can be a larger field of view for the surgeon performing the MIS.

According to some aspects of the disclosure, a system for use in a minimally invasive surgical procedure for providing direct stereoscopic views of a surgical area is provided. In particular the system can include: a percutaneous optical channel (POC) including a proximal end extending from the patient and a distal end extending towards a cavity above the surgical area; a retaining plug including at least one channel configured to hold said percutaneous optical channel (POC) through an incision proximate to the surgical area; and a stereoscopic optical device configured to provide to a practitioner at least one stereoscopic view of at least a portion of an internal surgical area via an optical path including said POC and said stereoscopic optical device.

According to other aspects of the disclosure, the system can include: a first percutaneous optical channel (POC) including a proximal end extending from the patient and a distal end extending towards a cavity above the surgical area and a second POC including a proximal end extending from the patient and a distal end extending towards a cavity above the surgical area, wherein said first POC and second POC are positioned through separate incisions in proximity to the surgical area and such that their field of views converge to an overlapping portion of said surgical area; and a stereoscopic optical device configured to provide to a practitioner at least one stereoscopic view of at least a portion of an internal surgical area via a plurality of optical paths including at least said first POC, second POC, and said stereoscopic optical device.

According to yet additional aspects of the disclosure, the system can include: a percutaneous optical channel (POC) including a proximal end extending from the patient and a distal end extending towards a cavity above the surgical area; a retaining plug including at least one channel configured to hold said percutaneous optical channel (POC) through an incision proximate to the surgical area; and a stereoscopic optical device configured to provide to a practitioner at least one direct stereoscopic view of at least a portion of an internal surgical area via an optical path including said POC and said stereoscopic optical device.

Other aspects of the invention will be understood by those of skill in the art upon review of the teachings herein. These other aspects of the invention may provide various combinations of elements from the various embodiments with various elements of the aspects presented above as well as provide other configurations, structures, functional relationships, and processes that have not been specifically set forth above.

### BRIEF DESCRIPTION OF THE DRAWINGS

FIG. 1 provides a cross-sectional view of elements of an exemplary visualization system in association with a surgical area under the surface of the skin and including a percutaneous optical channel (POC) according to some aspects of the disclosure.

FIG. 2A provides a cross-sectional view of an exemplary POC with annular protrusions according to some aspects of the present disclosure.

FIG. 2B provides a cross-sectional view of another exemplary POC with an additional optic lens according to some aspects of the present disclosure.

FIG. 2C provides a cross-sectional view of another exemplary POC with a light source on the distal end of the POC housing according to some aspects of the present disclosure.

FIG. 2D provides a cross-sectional view of another exemplary POC attached to an exemplary retaining plug according to some aspects of the present disclosure.

FIG. 3A provides a cross-sectional view of an exemplary retaining plug that may be used in conjunction with a POC according to some aspects of the present disclosure.

FIG. 3B provides a cross-sectional view of another exemplary retaining plug with a light source that may be used in conjunction with a POC according to some aspects of the present disclosure.

FIG. 3C provides a cross-sectional view of another exemplary retaining plug with auxiliary channels that can be sealed that may be used in conjunction with a POC according to some aspects of the present disclosure.

FIG. 3D provides a cross-sectional view of another exemplary inflatable or fillable retaining plug that may be used in conjunction with a POC according to some aspects of the present disclosure.

FIG. 4 provides a flowchart illustrating exemplary methods steps that can be implemented according to aspects of the present disclosure.

### DETAILED DESCRIPTION OF PREFERRED EMBODIMENTS

Some embodiments of the invention are directed to stereoscopic optical visualization systems for use in minimally invasive surgery (“MIS”) and their associated methods. In particular, these systems can include a plurality of lenses and possibly other optical elements (e.g. mirrors, filters, prisms and the like) which may be held together in a desired configuration (e.g. within a cylindrical or near-cylindrical housing), that may be fixed or adjustable, and can be inserted through and held in place relative to a small incision to act as a “peephole” into the body and are referred to herein as a percutaneous optical channel (“POC”). When coupled with an imaging device and display an indirect view of the area under surgery can be available to the surgeon. In an exemplary embodiment, a stereo camera, and a stereoscopic display stationary or head-mounted, (glasses free parallax barrier 3D display, etc.) may be located between the POC and the eyes of the surgeon to extract from the optical information provided through POC a stereoscopic image of the surgical area for presentation of the information through one or more display to a practitioner during minimally invasive surgery. According to some aspects of the disclosure, the display can be aligned with the camera in the sterile field as to display an ergonomically correct view of the surgical area to the surgeon.

In the present disclosure, a “direct” view or image can refer to an image that is directly presented from a source (e.g. a surgical area), that can provide a diffuse reflection of light, to the practitioner/surgeon with only intervening optical elements such as lenses, mirrors, prisms, filters, polarizers, variable apertures, and other strictly optical elements (e.g. other transparent optical elements), or the like without image capture elements (e.g. digital cameras or video recorders) and redisplay elements (e.g. electronic displays). For example, “direct” view can refer to a purely optical conveyance of information from a source to the eyes of a surgeon without conversion of the information into some other form (e.g. electronic data that is converted back into an optical image prior to reaching the eyes of the surgeon) converting the optical image into electronic data and back. In contrast, in the present disclosure an “indirect” view can refer to the conversion of an optical image to some other form and then back to an optical image. For example, an indirect image can have a break in the optical path wherein image capture occurs (e.g. by a digital camera or video recorder) and then conversion back into an optical form (e.g. by an electronic image display device—LED, LCD, plasma, CRT, or the like).

Depending on the area being viewed, the optical elements used in the POC, and the requirements of the particular procedure, the surgical view that can be provided to the surgeon may have different properties. For example, it may have a relatively narrow field of view (small solid angle) or relatively wide view of view (large solid angle), a relatively large depth of focus or a relative small depth of focus, it may reduce various optical aberrations down to a tolerable level, it may be filtered to remove or enhance certain wavelengths to provide enhanced viewing of a surgical area as a whole or of selected elements in the surgical area, or the like.

Accordingly, in some alternative embodiments additional views may be presented to the surgeon in addition to that presented from the POC. For example, in some embodiment variations, endoscopes and associated viewing instruments may provide additional views that may be either direct or indirect views. Accordingly, the surgeon may be provided with an indirect image captured from the POC that is then displayed without a significant delay. In some embodiments the display can be positioned in the sterile field as to provide a view that mimics open surgery.

According to some additional aspects of the disclosure, the electronically displayed images of the surgical area, as seen through the POC, can be supplemented by other electronically displayed images that are overlaid with the visual optical images to provide overlaid or composite images showing not only the visual content of the surgical area but also showing augmented content as well (e.g. shape of a relevant tissue region, distance of a surgical tool from a critical tissue surface, other data relevant to the surgical procedure, and the like). Further details about such augmented images and methods are set forth in co-pending US Patent Applications: (1) Ser. No. 13/164,671, filed Jun. 20, 201, and entitled “Augmented Reality Methods and Systems Including Optical Merging of a Plurality of Component Optical Images”; Ser. No. 13/169,072, filed Jun. 27, 2011, and entitled “Surgical Procedures Using Visual Images Overlaid with Visual Representations of Selected Three-Dimensional Data”; and Ser. No. 13/169,076, filed Jun. 27, 2011, and entitled “Surgical Procedures Using Instrument to Boundary Spacing Information Extracted from Real-Time Diagnostic Scan Data”. Each of these referenced applications is incorporated herein by reference as if set forth in full herein. According to some aspects of the disclosure, the electronically created or manipulated image may be overlaid onto the direct optical path being viewed by the surgeon via optical elements, such as partially reflective mirrors, prisms or other beam combining/splitting elements as discussed in the '671 application. Such enhanced embodiments provide the surgeon not only with a direct optical view but also with a simultaneous indirect optical view of additional information that may be useful to the surgeon during the surgical procedure.

According to other aspects of the disclosure, a system of optical lenses housed in a tube (e.g. a cylindrical tube or conical tube), called the percutaneous optical channel (POC), can be inserted into a main channel of a retaining plug. The POC may include proximal, intermediate, or distal features that help to retain the POC within the retaining plug or within the incision. Such retention features may be of a fixed configuration or variable configuration that aids in one or more of insertion, extraction, or retention. In some embodiment variations, the POC may be located in the retaining plug while the retaining plug is inserted into the incision, while in other embodiments it may be moved into the retaining plug after insertion of the retaining plug into the incision.

The length of the POC can be such that it can provide optical access though both ends of the retaining plug or the external tissue that it is penetrating. To accommodate the variations in retaining plug sizes or tissue thickness, POCs can be offered in a variety of different standard lengths, or a single POC may be adjustable in length (e.g. through telescopic action) to provide necessary accommodation. In some embodiments, the anterior or proximal side (relative to the surgeon) of the POC may have a socket for receiving additional lenses or other optical elements for adjusting the optical properties of the device for one or more different purposes, e.g. increasing or decreasing field of view, increasing or decreasing depth of view, increasing or decreasing magnification, providing optical filtering, and the like. In some embodiments the POC may be reusable while in others it may be a single use device. Accordingly, some POCs may be treated as disposable while others can be designed for multiple uses and the unit may be configured for ease of surface sterilizability or encapsulation by a disposable biocompatible encapsulating material.

In some embodiment variations, the POC and retaining plug combination is configured to be adjustable or replaceable during the surgical procedure to provide the surgeon with varying views, e.g. optimized views, of the surgical area as the surgery proceeds or as the surgeon's need for seeing certain features or elements changes.

Further in some embodiments, optional auxiliary pathways or channels in the optional retaining plug can be used for one or more of a variety of different purposes, e.g. for sending in light, for sending in fluids (e.g. gases or liquids), or extracting fluids (e.g. gases or liquids) or even small solids, and/or insertion of small instruments into the surgical cavity. Such channels may also be used, for example, as irrigation ports, aspiration ports, and insufflation ports. For example, after the POC is in into the main opening of the retaining plug, and if the auxiliary holes are also sealed off, or are in use as intended, the retaining plug may form an almost-airtight barrier, which may allow gases such as insufflated CO2, or liquids such as water, to be pumped into the surgical cavity to create a stable viewing and working area for the minimally invasive surgery.

During use of the POC, the surgical field can be illuminated in one or more of a variety of different manners, including, for example by (1) directing light from an outside light source through the POC into the body cavity; (2) directing light from an outside light source through an auxiliary hole in the retaining plug; (3) directing light from a separate instrument through a different incision/port; (4) from a miniature light source inserted into the surgical area (e.g. along with the POC or retaining plug or inserted via one of the auxiliary openings); (5) using lights which may be built into the POC housing in the form of a light ring or a string of individual lights around the distal rim of the POC; (6) using lights which may be built into the retaining plug in the form of a light ring or a string of individual lights around the distal rim of the retaining plug. In items (5) and (6) above, the light sources may be LED, fiber optic light rings, or light pipes among other light source options. In some embodiments the light or light sources may be directable or focusable in a manual, semiautomatic, or fully automatic manner. Fully or semiautomatic adjustments of lights or light sources may be implemented with the aid of one or more sensors that form part of the POC, part of the retaining plug, or that can be inserted separately. Such sensors may include, for example, light sensors and ultrasonic sensors. Adjustments may also be made using image processing techniques based on images provided by the imaging device. Such image processing may be used alone or in combination with semiautomatic or automatic, surgeon perceptible or imperceptible, perturbations of the light or light sources while comparing successive images for enhancements or degradations.

The posterior surface, or distal surface, of the POC (e.g. distal surface of the distal posterior lens) inserted into and facing the surgical cavity may be kept clean using a variety of different methods and associated mechanisms such as, for example: (1) directing a flow of a cleaning fluid such as saline water to flush and irrigate the surface, (2) covering the surface with a protective membrane or cap during the insertion phase, which can then be removed after the cavity is filled with gas or liquid as intended, (3) providing a wiper that can periodically sweep off the surface, (4) directing a flow of selected gas (e.g. CO2) across the surface. Liquids and/or gases may be directed to the distal surface via one of the other holes in the retaining plug, or through a different incision/port and may be extracted, as appropriate, through another hole in the retaining plug or through a different incision/port. In some embodiments, such a fluid may also be used to flush blood or other vision impeding tissues or materials from the surgical cavity. Cleaning of the distal end of the POC may occur via manual methods based on a surgeon's perception that cleaning would be useful, via semi-automatic or automatic methods where detection of a visual impediment occurs semi-automatically or automatically (e.g. via image quality reduction) which intern gives rise to a cleaning attempt or cleaning recommendation that the surgeon can then initiate.

In additional aspects of the disclosure, the image capturing device used for to provide an indirect view can include a stereo camera. The stereo camera may be in connection with a 3D display (eg. goggles, autostereoscopic display) and the POC being held by a retaining plug. The retaining plug can include at least one channel that is sealed by material via an elastic contact of sidewalls of at least one division wherein the sidewalls of the division can be separated to allow insertion of a desired tool or to allow passage of a desired material. The POC may be inserted into a retaining plug and thereafter into the at least one incision to cause substantial sealing of the incision around the lateral periphery of the at least one retaining plug. In some minimally invasive surgeries, it may be useful to include at least two POCs being inserted into separate incisions to observe the surgical area. In addition, in some system implementation, the visualization can include at least one augmented image generated from a non-visual source, converted into a visual display that can be aligned with and overlaid on the displayed visual images of the surgical area. The augmented image may include, for example, a plurality of augmented images that are updated periodically during the procedure.

Other objects and advantages of various embodiments of the invention will be apparent to those of skill in the art upon review of the teachings herein. The various embodiments of the invention, set forth explicitly herein or otherwise ascertained from the teachings herein, may address one or more of the above objects alone or in combination, or alternatively may address some other object ascertained from the teachings herein. It is not necessarily intended that all objects be addressed by any single embodiment or aspect of the invention even though that may be the case with regard to some embodiments or aspects.

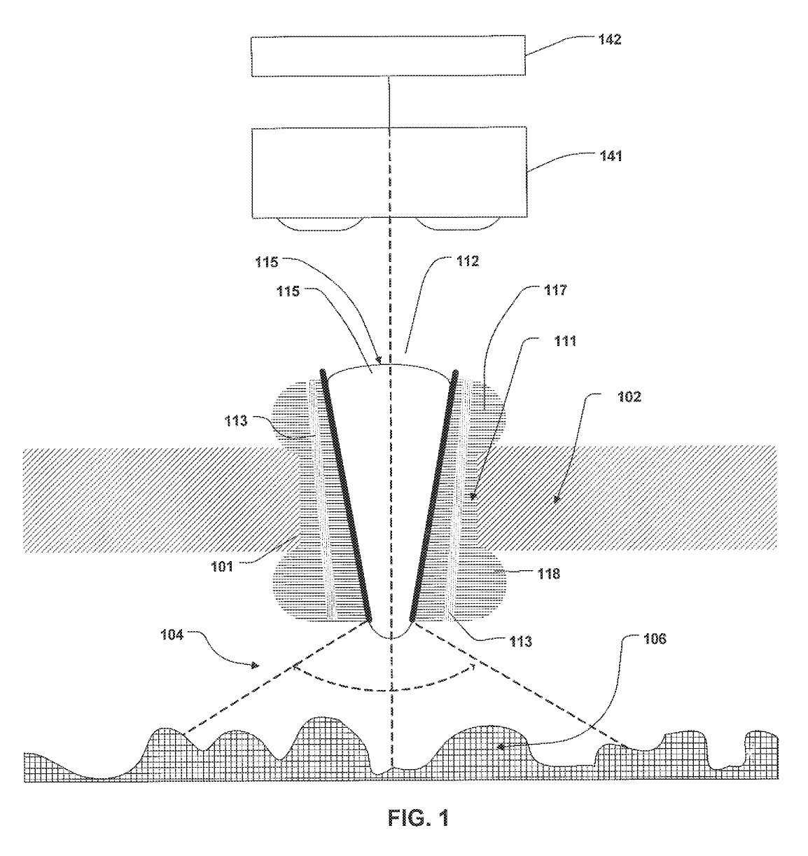

Referring now to FIG. 1, elements of an exemplary visualization system according to a first embodiment wherein elements of the visualization system are depicted. In particular, the elements are shown in association with a surgical area 106 that can be located under the surface of the skin and other tissue 102 of a patient. In this embodiment, the visualization system can include the POC 115 and an image capturing device 141. In addition, for improved viewing of a surgical area, light can be supplied to the surgical area, either from or through the POC itself, from or through a retaining plug 111 into which the POC is inserted, or from or through some other device that is inserted into the cavity 104. For example, the light source or illumination can include LEDs, fiber optics, or other light generating or directing sources, inserted into the cavity, incorporated into the POC, and/or retaining plug. In some embodiments, the light source may be part of the system while in other embodiments it may be considered a separate element or system that is necessary for desired optical information to be directed from the surgical area through the POC, through the additional optical system (e.g. stereo image acquisition unit) and then displayed to the eyes of the surgeon.

Referring back to FIG. 1, a small incision 101, usually less than 2 cm in length (but may be of any appropriate smaller or larger size), is made through the wall 102, e.g. through the skin and some underlying tissue 102 (e.g. fat and/or muscle) of a body cavity 104 that may be located or created over the intended surgery location (e.g. abdominal cavity, spine, hip, or knee, etc.). A “surgical retaining plug” 111 may be inserted into this incision 101 as shown. The retaining plug includes a main channel 112 through the middle for insertion of and/or holding of a POC 115, and may optionally contain one or more other auxiliary pathways or channels 113 to provide other functionality. (In alternative embodiment variations two or more channels may be used for holding retaining plugs and POCs but in the most preferred embodiments, a single POC and retaining plug is used in performing a minimally invasive procedure to reduce tissue trauma and recovery time.)

The retaining plug 111 may be designed to be capable of lateral expansion, i.e. expansion in a direction substantially perpendicular to the surface of the skin 102, to expand and push against the walls of the incision 101 after insertion. Furthermore, or alternatively, the retaining plug 111 may be configured to allow for lateral contraction for ease of removal and/or to ease tissue trauma. The retaining plug 111 may provide for an almost-airtight or watertight gateway to the surgical area during surgery when all of its channels 112, 113 are sealed. The length of the retaining plug may vary depending on the thickness of the tissue 102 it is intended to go through. The shape of the retaining plug 111 may include lateral protrusions 117 at the anterior end (i.e. the proximal end or end outside the patient) and/or lateral protrusions 118 at the posterior end (i.e. distal end or the end that extends into the cavity of the patient's body) that may help the retaining plug maintain a desired position (e.g. to keep it from easily slipping out during use). In some alternative embodiments, protrusions may be applied at one or more intermediate positions along the length of the retaining plug in addition to or lieu of the end protrusions. In still other embodiments, no protrusions may exist. Other methods can also be employed for this purpose, such as using sutures or clamps to firmly but temporarily attach the retaining plug to the skin or to other tissue. In some embodiment variations the retaining plug may be reusable while in others it may be a single use device. Accordingly, some retaining plugs may be treated as disposable while others can be designed for multiple uses and the unit may be configured for ease of surface sterilizability or encapsulation by a disposable biocompatible encapsulating material.

Referring back to FIG. 1, an image capturing device 141 and a display device 142 may be placed above the external (proximal) face of the POC 115, whereby the surgeon can view the images captured from the POC 115. Additional lenses or other optical elements, for example, may be placed in between the POC 115 and the image capturing device 141 for further optical magnification, image/color correction, and/or zooming purposes. Such additional optical elements may be configured to fit into a socket located on the proximal end of the POC. Alternatively, or in addition to, the further optical magnification, image/color correction, and/or zooming may be done digitally after the image is captured by the image capturing device 141.

In addition, as previously mentioned images viewed by the surgeon may be composite images of the surgical area as imaged through the POC in combination with electronically displayed images that have been overlaid. Such composite images may be obtained via electronic merging of the real-time visual images of the surgical area which are captured and sent to a computer to undergo electronic merging with electronically produced or manipulated augmented reality images. Such composite images may include visual images generated from 2-D and 3-D diagnostic scans, such as X-Rays, MRI, CT, etc., which are presented back to the surgeon via a computer display screen.

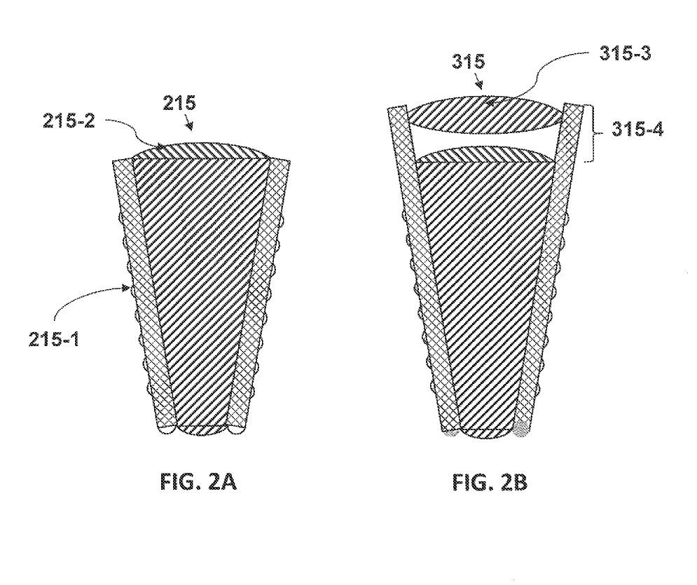

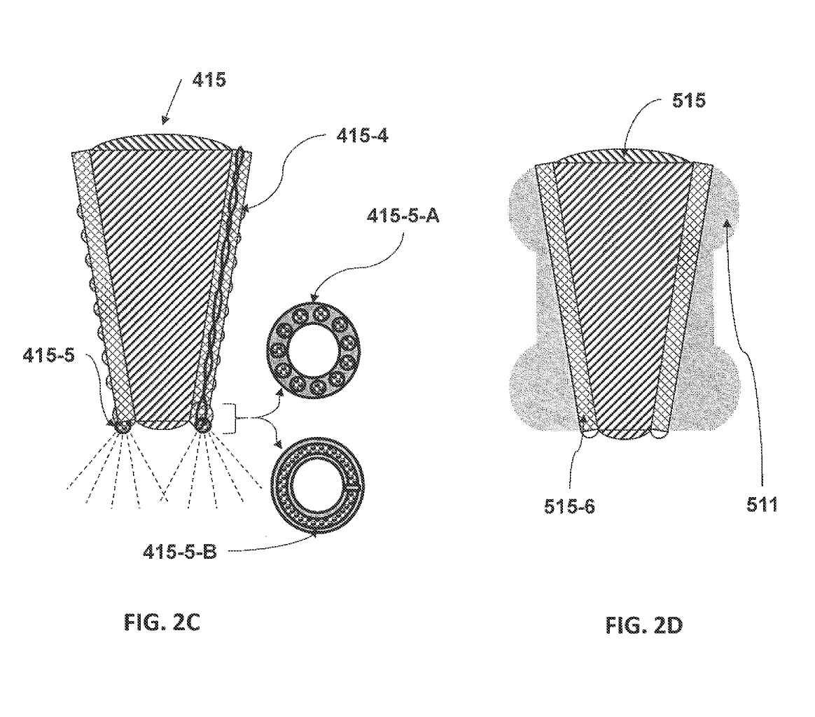

Referring now to FIG.2A, a side cross-sectional view of a POC 215 with one or more lenses 215-2 located within a housing 215-1 that holds the lenses together is illustrated. As shown, the housing may be provided with a plurality of bump-like protrusions or ring like protrusions that circle the modulate the exterior of the housing such surface texturing may help the POC resist slippage once it is placed in an incision or the retaining plug. In some embodiments, these bumps may take the shape of continuous spiral around the POC housing, such that they can be inserted into the retaining plug or released using screw turning action.

Referring now to FIG. 2B, a side cross-sectional view of another example POC 315 that includes a socket structure 315-4 on or near the proximal end to hold one or more additional optical elements (e.g. lens 315-3) is illustrated. In some embodiments, the one or more additional optical elements 315-3 may be installed independently for various optical image correction, enhancement, and/or zooming purposes. In some usages these additional optical elements may be installed prior to a surgical procedure while in other embodiments they may be inserted, removed and replaced during a surgical procedure while the POC and the retaining plug remain in place.

Referring now to FIG. 2C, a side cross-sectional view of another example POC 415 having one or more light sources 415-5 built into the housing is illustrated. As depicted, the light source(s) may take the form a single light ring 415-5-B or a pattern of multiple individual light elements 415-5-A. The light elements may include, for example, LEDs or miniature incandescent bulbs. Power may be supplied to the light source(s), for example, via wiring 415-4 that can be embedded in or that run along the surface of the housing. In some embodiments, the light intensity may be controllable via a variable voltage supply and in multiple light embodiments individual lights may be independently controllable. In alternative embodiments, the light may be non-electrical in nature but instead rely on another means for stimulating emission of radiation (e.g. chemical reaction, phosphorescence, or the like).

Referring now to FIG. 2D, a side cross-sectional view of another example POC 515 structure with built-in or already attached surgical retaining plug is illustrated. In particular, the POC may include structural retaining structures 515-6 that can prevent the POC from slippage after insertion through an incision. In some embodiments, the structural retaining features 511 on the distal end of may be inflatable or be made of soft materials that can be compressed and allow easy insertion through the incision. Other features described in other exemplary embodiments may be included accordingly. For example, auxiliary channels and/or one or more light sources can be included in similar embodiments.

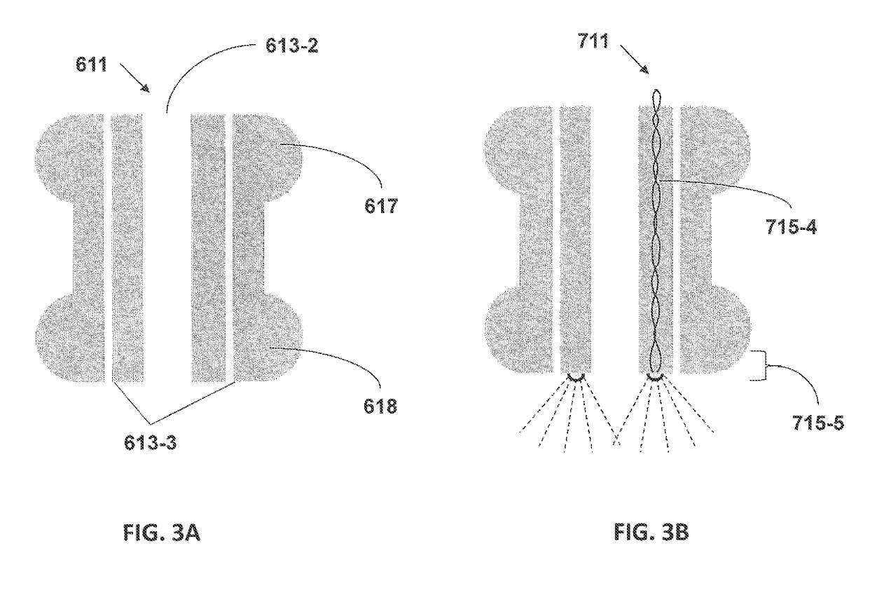

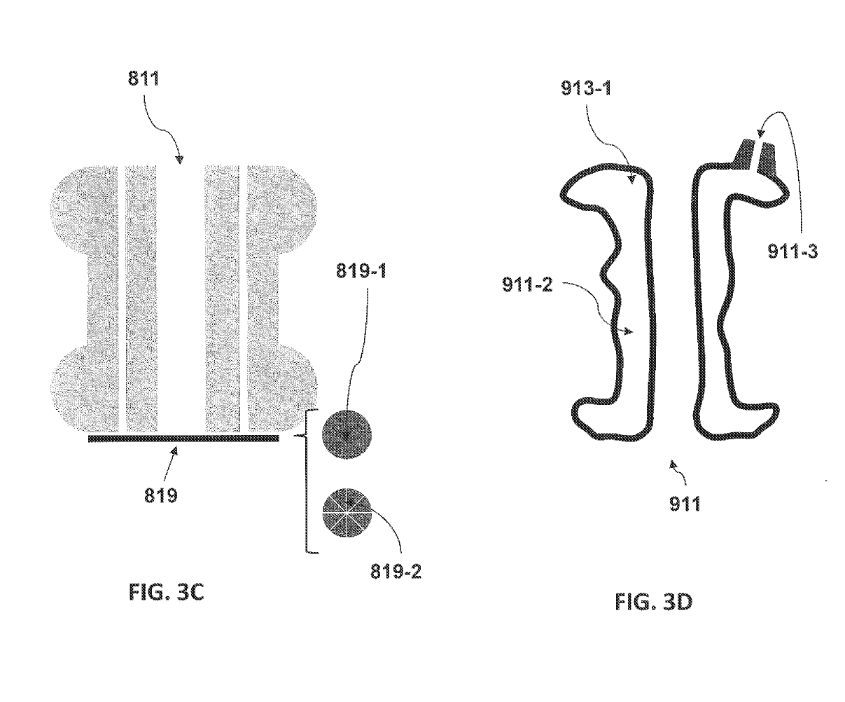

FIGS. 3A-3D provides side cross sectional views of four exemplary variations of retaining plugs. Each of these four exemplary retaining plugs may be inserted through an incision and fixed into place prior to POC insertion into their main channel, or they may be inserted into an incision with the POC already-attached as shown in FIG. 2D.

Referring now to FIG. 3A, a side cross-sectional view of an exemplary retaining plug 611 is illustrated. In particular, the retaining plug 611 can includes a main opening 613-2 for holding a POC and one or more auxiliary channels 613-3 (two are shown) that may be used for primarily for insufflation purposes. Other uses may be inserting and removing various tools or materials that would be of use during a surgical procedure. Tools and materials may include, for example, liquid for irrigation or aspiration, light, light sources, endoscopes, tissue manipulation instruments, etc. The retaining plug may be made from a variety of materials, including for example rigid plastics and other materials, sponge-like polymers, elastic, expandable, compressible, or flexible polymers or other materials. In order to insert a retaining plug into an incision, or to take a retaining plug out of an incision, the surgeon may have to shrink its lateral profile by squeezing or distorting it with a grasper. In some embodiments, plugs may be formed from multiple materials, (e.g., some substantially rigid and some elastically deformable), each which may provide a useful property to the retaining plug as a whole.

Referring now to FIG. 3B, a side cross-sectional view of another example surgical retaining plug 711 is illustrated. In particular, the retaining plug 711 can include built in lights 715-5 and wiring 715-4 similar to lights 415-5 and wiring 415-4 of FIG. 2C but this time with the light forming part of the retaining plug. As with the lights of FIG. 2C, the lights in this embodiment actually be a single light source or may be multiple light sources. The other variations noted with regard to FIG. 2C, mutatis mutandis, also apply to this retaining plug embodiment.

Referring now to FIG. 3C, a side cross-sectional view of another examplary surgical retaining plug 811 is illustrated. In particular, the opening or channels are sealed with one or more membranes 819 located at the distal end. The membrane(s) 819-1 may be airtight, in which case it or they may be torn or popped open during the insertion of the POC or other instruments. Alternatively, the retaining plug's 811 channels may have flaps 819-2 that bend and open during insertions of the POC or other instruments or materials. In yet additional embodiments, the membranes over the auxiliary channels may actually be self-sealing septums that allow passage of necessary tools or material but provide for closure upon removal of the tool, instrument, or material transfer. For example, the auxiliary channels, and even the main channel, may be filled in whole or in part with a compressible sealing material that includes one or more divisions with either side of the division being pressed against the other side via elastic forces wherein insertion of tools or material can be made to occur by temporarily overcoming the elastic force to cause parting along the division wherein the barrier material is sufficiently elastic and durable to reseal after tool is removed or material passage completed.

Referring now to FIG. 3D, a side cross-sectional view of another example surgical retaining plug 911 is illustrated. In particular, the retaining plug 911 can have walls with hollow interiors 911-2 and an inflation or filing port 911-3 that can be used such that during insertion and removal of the retaining plug it can be deflated, and after insertion it is inflated or filled to laterally expand and cause sealing against the side walls of the incision. In some variations, only portions of the walls of the retaining plug need to be inflatable or fillable.

The features of the first embodiment, the variations thereof, and the variations set forth in FIGS. 2A-2D and 3A-3D may be mixed and matched as appropriate to derive numerous embodiments that have features that are tailored to a given set of circumstances (e.g. surgical or financial). Some such feature combinations may remove elements without replacing them while others may replace the removed elements with alternative elements, while still other combinations will simply add elements.

In accordance to some aspects decribed, the POC examples with or without the retaining plug will be useful for providing enhanced images of a surgical area in a variety of minimally invasive surgical procedures including for example: (1) laparoscopic cholecystectomy, (2) appendectomy, (3) nissen fundoplication, (4) gastric band, (5) hysterectomy, etc. For laparoscopic surgeries for example, significant utility will be found in cholecystectomy, hernia repair, bariatric procedures (bypass, banding, sleeve, or the like), bowel resection, hysterectomy, appendectomy, gastric/anti-reflux procedures, and nephrectomy. In addition to using aspects of the disclosure on humans for the aforementioned procedures, the teachings of the disclosure can also be used for in vivo testing, animal clinical research and the such.

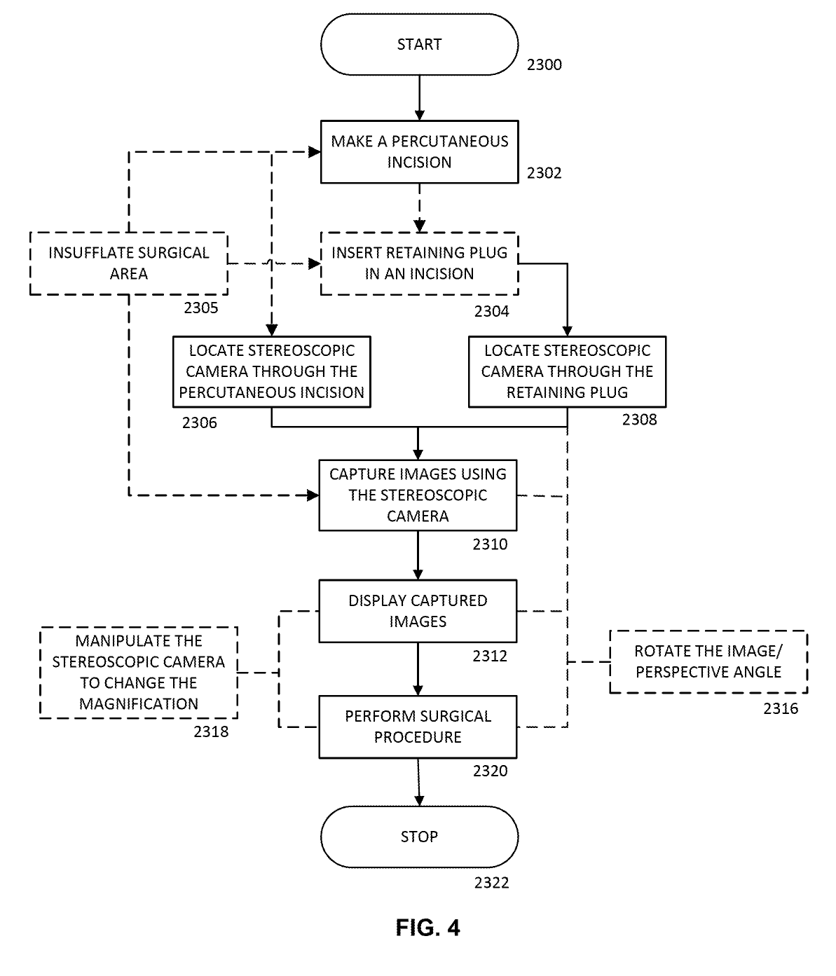

Referring now to FIG. 4, a flowchart illustrating exemplary method steps that can be implemented according to aspects of the present disclosure are shown. Beginning at step 2300, sterilization and/or any other commonly known and performed routine to begin a MIS procedure may occur. Subsequently, at step 2302, a percutaneous incision in the skin of a patient can be made. As previously described, the incision may be made around an area where the MIS procedure will take place. The size of the percutaneous incision can be so that a retaining plug can be tightly inserted through the skin.

In some embodiments of the system, optionally at step 2304, a retaining plug can be inserted through the percutaneous incision. At 2305, before, during or after the insertion through the percutaneous incision of step 2304, the surgical are may be insufflated to allow the MIS to take place. As previously explained, one functional purpose of the retaining plug can include holding the device down to the patient by an expanded flange. Some retaining plugs may be deformable enough to allow insertion into the incision, either by the natural compliance of the material that it is constructed from, by being or having inflatable components, or having articulating components. In some embodiments the retaining plug may be disposable, but at the minimum it should be sterilizable. Also previously described, in some embodiments such as FIG. 2D, the POC may include structural features or articulating components capable of holding the stereoscopic camera onto the patient. In these types of embodiments, at step 2306, at least a portion of the objective forming part of the stereoscopic camera may be inserted through the percutaneous incision without the need of a retaining plug. The objective lens or lens assembly 103 being inserted may be made of glass or plastic, however in some preferred embodiments it can be disposable, but at the minimum it should be sterilizable.

In embodiments where a retaining plug is used, at step 2308, at least a portion of the image capturing device can be located through the retaining plug. At step 2310, images can be captured using the image capturing device, e.g. a stereoscopic camera. Processing of the captured images can then occur for a processor to display the captured images at step 2312. The display however may also have touch screen controls for zoom, focus, image freezing, or other camera mode selections. A touch screen interface could be button based or gesture based. For example, a gesture to zoom out would be to perform a two finger pinching motion on the screen and the picture-in-picture roles could be reversed by swiping from the smaller image to the center of the screen. The display 701 may support VGA resolution (640x480) all the way up to true high definition (1920×1080p) or beyond. Since the image capturing device can be stereoscopic, the display preferably supports either active or passive 3D display technology. In the some embodiments, the display is autostereoscopic (e.g. parallax barrier), requiring no glasses for viewing a 3-D effect.

Referring back to FIG. 23, at step 2320, the MIS procedure can then be performed by the practitioner utilizing the stereoscopic visualization system. At a point prior to, during, and/or after the MIS procedure, steps 2318, and/or 2316 may take place as it may be appropriate. However, one or more of these steps may not occur depending on the type of MIS procedure, and the settings and configuration of the embodiment being implemented.

At step 2316, the image/perspective angle may be rotated as previously described. For example, the image may be rotated relative to the acquisition direction to transform it to the viewing orientation of the surgeon. An image processing computer may take commands from the surgeon so as to provide one or more selected views with orientations or perspectives that can be different from that originally captured by the image capturing device.

At step 2318, the stereoscopic camera and/or an associated component can be manipulated to change the magnification. For example, the video acquisition unit can typically include optical zoom and focusing mechanisms, photosensitive integrated circuits 204, and digital image processing electronics which can be manipulated/adjusted. Moreover, in some embodiments, two photosensitive integrated circuits, one associated with each pupil, and thus with each optical channel can be created by the two stereoscopic pupils 203, may be the extent of the electronic components in the unit. However, to get better image quality and truer color, 3 or 4 photosensitive integrated circuits may be used to sense different wavelengths of light separately (e.g. red, green, and blue). In this case, extra optical hardware may need to be added, such as dichroic prisms, in order to optically separate the different wavelengths of light. In still other embodiment variations, it may be desirable to sacrifice image quality for compactness, and use a single photo sensor to capture both right and left images, half for the left and half for the right. Zooming may be continuous, or could have a finite number of discrete zoom levels. Focus may be manual or automatic.

At step 2322, after the MIS procedure is finished, the retaining plug and/or stereoscopic camera may be removed from the percutaneous incision. It is to be understood that an additional number of steps can occur depending on the embodiments as well as the type of MIS procedure.

The many features and advantages of the invention are apparent from the detailed specification, and thus, it is intended by the appended claims to cover all such features and advantages of the invention which fall within the true spirit and scope of the invention. Further, because numerous modifications and variations will readily occur to those skilled in the art, it is not desired to limit the invention to the exact construction and operation illustrated and described, and accordingly, all suitable modifications and equivalents may be resorted to, falling within the scope of the invention.

We claim:

1. A method of providing stereoscopic views of a surgical area during a minimally invasive surgical procedure, the method comprising:
providing an image through a percutaneous optical channel (POC) including a proximal end extending from the patient and a distal end extending towards a cavity above the surgical area;
configuring a retaining plug to hold said percutaneous optical channel (POC) through an incision proximate to the surgical area, wherein the retaining plug includes at least one protrusion substantially perpendicular to the optical channel; and
further configuring a stereoscopic optical device to provide to a practitioner at least one stereoscopic view of at least a portion of an internal surgical area via an optical path including said POC and said stereoscopic optical device.

2. The method of claim 1, wherein the retaining plug comprises at least one auxiliary channel configured to provide a passageway for one or more of: tools, light, objects, liquids, and gases during a minimally invasive surgery.

3. The method of claim 1, additionally comprising:
illuminating the surgical area via a light source included forming part of said retaining plug.

4. The method of claim 1, additionally comprising:
illuminating the surgical area via a light source included forming part of said POC.

5. The method of claim 1, additionally comprising:
securing said POC and retaining plug onto the incision proximate to the surgical area using retaining features forming part of the retaining plug.

6. A method of providing stereoscopic views of a surgical area during a minimally invasive surgical procedure, the method comprising:
providing a first percutaneous optical channel (POC) including a proximal end extending from the patient and a distal end extending towards a cavity above the surgical area and a second POC including a proximal end extending from the patient and a distal end extending towards a cavity above the surgical area, wherein said first POC and second POC are positioned through separate incisions in proximity to the surgical area and such that their field of views converge to an overlapping portion of said surgical area; and
configuring a stereoscopic optical device to provide to a practitioner at least one stereoscopic view of at least a portion of an internal surgical area via a plurality of optical paths including at least said first POC, second POC, and said stereoscopic optical device.

7. The method of claim 6, wherein the stereoscopic optical device is configured to digitally capture images of the internal surgical area.

8. The method of claim 7, wherein the digitally captured images of the internal surgical area are combined to provide a stereoscopic image using a display in the sterile field and in connection with said stereoscopic optical device.

9. The method of claim 8, additionally comprising:
overlaying at least a portion of an image onto the digitally captured images before displaying them on said display in the sterile field.

10. The method of claim 8, additionally comprising:
modifying the displaying of said digitally captured images based on a practitioners input.

11. The method of claim 10, wherein the modification includes one or more of: changing a magnification of an image, a viewpoint angle, a number of images, and a contrast of the digitally captured images.

12. The method of claim 11, additionally comprising:
configuring one or more retaining plugs to hold one or both of said first and second POCs through each of said separate incisions proximate to the surgical area.

13. The method of claim 12, additionally comprising:
providing a light source forming part of at least one of said retaining plugs associated with said first and said second POCs configured to illuminate at least part of the surgical area.

14. The method of claim 12, wherein at least one of said retaining plugs includes at least one auxiliary channel configured to provide a passageway for one or more of: tools, light, objects, liquids, and gases during a minimally invasive surgery.

15. The method of claim 12, wherein each of said retaining plugs comprises at least one protrusion extending in a direction substantially perpendicular to an optical axis of the POCs.

16. A method for use in a minimally invasive surgical procedure for providing indirect optical stereoscopic views of a surgical area, comprising:
providing a percutaneous optical channel (POC) including a proximal end extending from the patient and a distal end extending towards a cavity above the surgical area; and
configuring a stereoscopic optical device in communication with a display in the sterile field for providing, to a practitioner, at least one indirect stereoscopic view of at least a portion of an internal surgical area.

17. The method of claim 16, wherein the stereoscopic optical device is configured to digitally capture images of the internal surgical area.

18. The method of claim 17, wherein the digitally captured images of the internal surgical area are combined to provide a stereoscopic image using a display in the sterile field and in connection with said stereoscopic optical device.

19. The method of claim 18, additionally comprising:
overlaying at least a portion of an image onto the digitally captured images before displaying them on said display in the sterile field.

20. The method of claim 18, additionally comprising:
modifying the displaying of said digitally captured images based on a practitioners input.

21. The method of claim 20, wherein the modification includes one or more of: changing a magnification of an image, a viewpoint angle, a number of images, and a contrast of the digitally captured images.

22. The method of claim 16, additionally comprising:
configuring one or more retaining plugs to hold said POCs through an incision leading to said cavity above the surgical area.

23. The method of claim 22, additionally comprising:
providing a light source forming part of said retaining plug associated with said POC configured to illuminate at least part of the surgical area.

24. The method of claim 22, wherein said retaining plug includes at least one auxiliary channel configured to provide a passageway for one or more of: tools, light, objects, liquids, and gases during a minimally invasive surgery.

25. The method of claim 24, wherein each retaining plug comprises at least one protrusion extending in a direction substantially perpendicular to an optical axis of the POC.

26. The method of claim 24, additionally comprising:
insufflating, through said at least one of the auxiliary channels, the patient after the insertion of said POC into the retaining plug.
